(19) United States
(12) Patent Application Publication
(10) Pub. No.: US 2026/0209275 A1
(43) Pub. Date: Jul. 23, 2026

(54) PEPTIDE(S), COMPOSITION(S) AND METHOD(S) HAVING BROAD SPECTRUM ACTIVITY AGAINST CROP INSECTS

(71) Applicant: TELLURIS BIOTECH INDIA PRIVATE LIMITED, Hyderabad, Telangana (IN)

(72) Inventors: Alejandro CALDERONURREA, Fresno, CA (US); Venu POLINENI, Hyderabad, Telangan (IN); Yogiraj ZIRWAL, Nashik, Maharashtra (IN); Glenda W. POLACK, Fresno, CA (US)

(73) Assignee: TELLURIS BIOTECH INDIA PRIVATE LIMITED, Hyderabad, Telangana (IN)

(21) Appl. No.: 19/139,630

(22) PCT Filed: Dec. 15, 2023
(86) PCT No.: PCT/IN2023/051190

§ 371 Date: Jun. 16, 2025

(30) Foreign Application Priority Data
Dec. 15, 2022 (IN) 202241072527

(51) Int. Cl. C07K 7/08; A01N 37/46; A01P 7/04; C12N 1/20; C12N 15/70; C12R 1/19

(52) U.S. Cl. CPC C07K 7/08; A01N 37/46; A01P 7/04; C12N 1/20; C12N 15/70; C07K 2319/21; C12R 2001/19

(57) ABSTRACT

The present disclosure provides broad spectrum peptide(s) and compositions comprising the same that allow effective management of crop pests. Associated method(s) employing the said peptide(s) and compositions for the management of crop pests are also provided. The peptides of the present disclosure possess low likelihood of resistance development in pests and may be produced by methods that are more economically viable as compared to existing technologies.

### TECHNICAL FIELD

The present disclosure relates to the field of insect or pest management and crop improvement. Particularly, the present disclosure provides broad spectrum peptide(s) and compositions comprising the same that allow effective management of crop pests. Associated method(s) employing the said peptide(s) and compositions for the management of crop pests are also provided.

### BACKGROUND OF THE DISCLOSURE

Insect pests are known to cause damages to crops. The economically significant orders of pests are: Lepidoptera, Hemiptera, Diptera, Coleoptera and Thysanoptera. Different orders have different biology so most of existing control mechanisms are limited to pests falling in one order or many a times specific to pest species of an order. There are presently no control mechanisms that can effectively target all the important pests falling under the different orders.

Currently available technologies fall under the broad categories of biopesticides of plant or microbial origin, transgenic control, and chemically synthesized molecules. Challenges faced in these categories include supply constraints of raw materials, difficulty of isolation and stabilization and potential toxicity to beneficial insects when biopesticides of plant origin are considered. Those of microbial origin often demonstrate non-specificity or are limited by strain sensitivity. Transgenic control experiments are time taking, cost intensive and tend to find non-acceptance in the community, accompanied by stringent regulatory processes. Chemically synthesized molecules tend to be effective against fewer insects with higher possibility of resistance development in a short span of time. Moreover, like plant based biopesticides, although effective towards the target, these molecules generally affect non-target beneficial insects and microbes and are not entirely friendly to environment. The development pipeline of these molecules tends to be long and therefore the synthesis and production tend to be cost intensive.

Taken together, there is a need for broad spectrum agents possessing efficacy against crop pests, without posing a threat to the environment and overcoming other challenges of existing technologies as identified above.

### SUMMARY OF THE DISCLOSURE

The present disclosure provides means to control major crop pests across the different orders of the class Insecta.

Particularly, the present disclosure provides a peptide or combination of peptides selected from a group comprising sequences represented by SEQ ID Nos. 1-3 and 7-9, wherein the peptide or combination of peptides show broad-spectrum activity against crop pests.

In an exemplary embodiment, when the peptide is selected from a group comprising sequences represented by SEQ ID Nos. 1-3, it is in combination with one or more of the rest of SEQ ID Nos. 1-3 and 7-9.

In some embodiments, the peptide is selected from a group comprising sequences represented by SEQ ID Nos. 7-9; and wherein the combination of peptides comprises peptides selected from a group comprising sequences represented by SEQ ID Nos. 1-3 and 7-9.

In some embodiments, the combination of peptides comprises at least two or at least three peptides selected from a group comprising sequences represented by SEQ ID Nos. 1-3 and 7-9.

Further provided in the present disclosure are nucleotide sequence(s) encoding the peptide or combination of peptides as described above.

In some embodiments, the nucleotide sequence(s) are selected from a group comprising sequences represented by SEQ ID Nos. 4, 5, 6, 10, 11 and 12.

Further provided herein are vector(s) comprising the nucleotide sequence(s) as described above.

The present disclosure also provides a host cell comprising the vector(s) as described above, wherein the host cell is a prokaryotic or a eukaryotic cell.

Further envisaged herein is a composition comprising the peptide or combination of peptides of the present disclosure, optionally along with additive(s).

In some embodiments, the composition comprises about 1% (v/v) to about 75% (v/v) of the peptide or combination of peptides and about 25% (v/v) to about 99% (v/v) of the additive(s) In some embodiments, the additive(s) in the composition is selected from a group comprising extender, emulsifier and/or surfactant stabilizers, synergist, dispersant, solvents, thickeners, penetrants, antidraft agents, antifoam agents, antimicrobial agents, preservative, antifreeze agents or any combination thereof.

The present disclosure further provides a method for controlling crop pests or insects comprising application of the peptide or combination of peptides or the composition as described above.

In some embodiments, the application is performed by a method selected from a group comprising drip, drench, foliar application, broadcasting, dusting and seed coating or any combination thereof.

In some embodiments, the aforesaid method confers a crop pest or insect mortality rate of about 15% to about 100% and/or a population reduction in the next generation by at least 30%.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with detailed description below, are incorporated in and form part of the specification, and serve to further illustrate the embodiments and explain various principles and advantages, in accordance with the present disclosure where:

FIG. 1 depicts the number of Aphids in cotton crop 1, 3 and 9 days after application of the composition of the present disclosure comprising the peptide of Seq Id no. 3 and additives.

FIG. 2 depicts the number of Aphids in cotton crop 1, 3 and 9 days after application of the composition of the present disclosure comprising the peptides of Seq Id nos. 1 and 3 and additives.

FIG. 3 depicts the number of Thrips in chilli plant 1 and 3 days after application of the composition of the present disclosure comprising the peptides of Seq Id nos. 1 and 2 and additives.

### DETAILED DESCRIPTION OF THE INVENTION

Addressing the aforesaid need pertaining to broad spectrum agents possessing efficacy against crop pests, the present disclosure provides means for efficient management of crop pests/insects.

### Definitions

Unless defined otherwise, all technical and scientific terms used herein have the same meaning as commonly understood by one of ordinary skill in the art to which this invention belongs.

Although any methods and materials similar or equivalent to those described herein can be used in the practice or testing of the present invention, the preferred methods and materials are now described. Several terms are defined and used throughout the specification with the following definitions provided for convenience.

The term “crop pests” in the context of the present disclosure refers to insects/pests known to infect and/or damage plants.

Reference to “% similarity” or equivalent terms/phrases in the present disclosure, in reference to defined peptides, indicates a percentage of amino acid residues in the candidate sequence that are identical with the residues of a corresponding parent peptide, after aligning the sequences. Said phrase, when used in reference to a nucleotide sequence, apart from modifications to the nucleic acid sequence, envisages modifications in the codons that do not lead to a change in the amino acids encoded by the codons. Thus, by using the term “% similarity” and other like forms, it is to be understood that any molecule, whether nucleic acid or peptide, that functions similarly, and/or contains sequence identity, and/or is conserved structurally so that it approximates the reference sequence by the defined % identity is to be considered as part of this invention.

In the context of the present disclosure 85% sequence similarity covers about 85%, about 86%, about 87%, about 88%, about 89%, about 90%, about 91%, about 92%, about 93%, about 94%, 5 about 95%, about 96%, about 97%, about 98%, about 99% or about 100% identity to the parent peptide.

The term “derivative” in the context of the present disclosure is used in reference to modifications to the parent peptide, retaining at least 85% sequence similarity to the parent peptide. Said modifications include but are not limited to amino acid sequence such as addition, substitution, deletion and glycosylation variants, N-/C-terminal extensions, covalent modifications of a native polypeptide, encapsulation of a polypeptide or conjugation of the polypeptide to another substance or component, without substantially changing the function of the sequence.

The term “nucleotide” or “nucleotide sequence” assumes ordinary meaning of the said term that is conventionally used and well known to a person skilled in the art. It refers to any one or more nucleic acid segments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression ‘at least’ or ‘at least one’ suggests the use of one or more elements or ingredients or quantities, as the use may be in the embodiment of the disclosure to achieve one or more of the desired objects or results. Numerical ranges stated in the form ‘from x to y’ include the values mentioned and those values that lie within the range of the respective measurement accuracy as known to the skilled person. If several preferred numerical ranges are stated in this form, of course, all the ranges formed by a combination of the different end points are also included.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, the terms “include” (any form of “include”, such as “include”), “have” (and “have”), “comprise” etc. any form of “having”, “including” (and any form of “including” such as “including”), “containing”, “comprising” or “comprises” are inclusive and will be understood to imply the inclusion of a stated element, integer or step, or group of elements, integers or steps, but not the exclusion of any other element, integer or step, or group of elements, integers or steps

It is to be noted that the term “a” or “an” entity refers to one or more of that entity. As such, the terms “a” (or “an”), “one or more”, and “at least one” can be used interchangeably herein.

As regards the embodiments characterized in this specification, it is intended that each embodiment be read independently as well as in combination with another embodiment. For example, in case of an embodiment 1 reciting 3 alternatives A, B and C, an embodiment 2 reciting 3 alternatives D, E and F and an embodiment 3 reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

### DISCLOSURE

### Peptides of the Present Disclosure and Combinations Thereof

The present disclosure provides means to control major crop pests across the different orders of the class Insecta. In some embodiments, the peptides show broad spectrum activity in the management of crop pests.

Particularly, the present disclosure provides one or more peptides selected from a group comprising sequences represented by SEQ ID Nos. 1, 2 and 3 or derivatives thereof. Non-limiting examples of derivatives of the sequences represented by SEQ ID Nos. 1, 2 and 3 include sequences bearing at least about 90% sequence similarity to the said sequences.

Thus, in some embodiments, envisaged herein are one or more peptides selected from a group comprising SEQ ID Nos. 1, 2 and 3. In some embodiments, envisaged herein are one or more peptides bearing at least about 90% sequence similarity to sequence(s) selected from a group comprising SEQ ID Nos. 1, 2 and 3. In some embodiments, said one or more peptides bearing at least 90% sequence similarity to SEQ ID Nos. 1, 2 and 3 are represented by SEQ ID Nos. 7, 8 and 9. Accordingly, further envisaged in the present disclosure are one or more peptides selected from a group comprising SEQ ID Nos. 7, 8 and 9.

Accordingly, in some embodiments, the present disclosure provides a peptide represented by Seq ID No. 1 and derivatives thereof. In some embodiments, the present disclosure provides a peptide represented by Seq ID No. 2 and derivatives thereof. In some embodiments, the present disclosure provides a peptide represented by Seq ID No. 3 and derivatives thereof.

In some embodiments, the peptides represented by SEQ ID Nos. 1-3 are isolated peptides or synthetic peptides.

In an exemplary embodiment, the peptides represented by SEQ ID Nos. 1-3 are isolated peptides. Accordingly, in some embodiments, the present disclosure provides isolated peptides selected from a group comprising peptides represented by SEQ ID Nos. 1-3.

In some embodiments, the present disclosure provides a peptide represented by Seq ID. 7. In some embodiments, the present disclosure provides a peptide represented by Seq ID. 8. In some embodiments, the present disclosure provides a peptide represented by Seq ID. 9.

In some embodiments, the peptides represented by SEQ ID Nos. 7-9 are synthetic peptides. Accordingly, in some embodiments, the present disclosure provides synthetic peptides selected from a group comprising peptides represented by SEQ ID Nos. 7-9.

In some embodiments, the present disclosure provides a combination of peptides selected from a group comprising sequences represented by SEQ ID Nos. 1-3 and peptides bearing at least 90% sequence similarity thereto.

In some embodiments, the present disclosure provides a combination of peptides selected from a group comprising sequences represented by SEQ ID Nos. 1-3 and 7-9.

In some embodiments, the present disclosure provides a combination of two or more peptides selected from SEQ ID Nos. 1-3. In some embodiments, the present disclosure provides a combination of two or more peptides selected from SEQ ID Nos. 7-9.

In some embodiments, the present disclosure provides a combination of peptides comprising two or more peptides selected from a group comprising sequences represented by SEQ ID Nos. 1-3 and 7-9. In some embodiments, the present disclosure provides a combination of peptides comprising three or more peptides selected from a group comprising sequences represented by SEQ ID Nos. 1-3 and 7-9. In some embodiments, the present disclosure provides a combination of peptides comprising at least four or at least five peptides selected from a group comprising sequences represented by SEQ ID Nos. 1-3 and 7-9.

Each of the above defined peptides independently and in combination with each other show broad-spectrum activity against crop pests.

Accordingly, the present disclosure provides a peptide or combination of peptides selected from a group comprising sequences represented by SEQ ID Nos. 1-3 and 7-9, wherein the peptide or combination of peptides show broad-spectrum activity against crop pests.

In some embodiments, the peptide is selected from a group comprising sequences represented by SEQ ID Nos. 1-3; and the combination of peptides comprises two or more peptides selected from a group comprising sequences represented by SEQ ID Nos. 1-3 and 7-9.

In an exemplary embodiment, the peptide is selected from a group comprising sequences represented by SEQ ID Nos. 7-9; and the combination of peptides comprises two or more peptides selected from a group comprising sequences represented by SEQ ID Nos. 1-3 and 7-9.

In some embodiments, provided herein is a peptide or combination of peptides selected from a group comprising sequences represented by SEQ ID Nos. 1-3 and 7-9, wherein the peptides represented by SEQ ID Nos. 1-3 are mandatorily in combination with at least one of the rest of SEQ ID Nos. 1-3 and 7-9. Accordingly, in some embodiments, when the combination of peptides comprises any of SEQ ID Nos. 1-3, the combination further comprises one or more of the rest of SEQ ID Nos. 1-3 and 7-9.

In some embodiments, the combination of peptides comprises at least two or at least three peptides selected from a group comprising sequences represented by SEQ ID Nos. 1-3 and 7-9.

In some embodiments, the peptide(s) of the present disclosure may further comprise a histidine tag.

Table 1 below shows the peptides of the present disclosure and their respective sequences, wherein the amino acids in bold depict the sequence variations between the peptides.

TABLE 1
Peptides and their respective sequences

| Peptides | Amino acid sequence | Corresponding Nucleotide sequence |
|---|---|---|
| SEQ ID No. 1 | MAVAFYIPDQATLLR | ATGGCGGTGGCCTTTTATATCCCGGATCAGGCAACCCTC |
| | | TTACGT [SEQ ID No. 4] |
| SEQ ID No. 1 with | MAVAFYIPDQATLLRHHHHHH | ATGGCGGTGGCCTTTTATATCCCGGATCAGGCAACCCTC |
| His-tag | | TTACGTCATCATCATCACCACCAC |
| SEQ ID No. 2 | MWRFLATVVLETLRQ | ATGTGGCGTTTTCTGGCGACGGTCGTGCTGGAAACCCTG |
| | | CGCCAG [SEQ ID No. 5] |
| SEQ ID No. 2 with | MWRFLATVVLETLRQHHHHHH | ATGTGGCGTTTTCTGGCGACGGTCGTGCTGGAAACCCTG |
| His-tag | | CGCCAGCATCATCACCACCACCAT |
| SEQ ID No. 3 | MAEQKEQQILRLRES | ATGGCAGAACAAAAAGAACAACAGATTCTGCGTCTGCG |
| | | TGAATCC [SEQ ID No. 6] |
| SEQ ID No. 3 with | MAEQKEQQILRLRESHHHHHH | ATGGCAGAACAAAAAGAACAACAGATTCTGCGTCTGCG |
| His-tag | | TGAATCCCATCATCACCACCACCAT |
| SEQ ID No. 7 | MAVAFYIPDQATLIK | ATGGCGGTGGCCTTTTATATCCCGGATCAGGCAACCCTC |
| | | ATTAAA [SEQ ID No. 10] |
| SEQ ID No. 7 with | MAVAFYIPDQATLIKHHHHHH | ATGGCGGTGGCCTTTTATATCCCGGATCAGGCAACCCTC |
| His-tag | | ATTAAACATCATCATCACCACCAC |
| SEQ ID No. 8 | MWRFLATVVLDTLKQ | ATGTGGCGTTTTCTGGCGACGGTCGTGCTGGATACCCTG |
| | | AAGCAG [SEQ ID No. 11] |
| SEQ ID No. 8 with | MWRFLATVVLDTLKQHHHHHH | ATGTGGCGTTTTCTGGCGACGGTCGTGCTGGATACCCTG |
| His-tag | | AAGCAGCATCATCACCACCACCAT |
| SEQ ID No. 9 | MAEQKEQQILRLRDT | ATGGCAGAACAAAAAGAACAACAGATTCTGCGTCTGCG |
| | | TGACACA [SEQ ID No. 12] |
| SEQ ID No. 9 with | MAEQKEQQILRLRDTHHHHHH | ATGGCAGAACAAAAAGAACAACAGATTCTGCGTCTGCG |
| His-tag | | TGACACACATCATCACCACCACCAT |

As can be seen from the above table, SEQ ID No. 7 is a derivative of SEQ ID No. 1; SEQ ID No. 8 is a derivative of SEQ ID No. 2; and SEQ ID No. 9 is a derivative of SEQ ID No. 3.

### Nucleotides Encoding the Peptide or Combination of Peptides of the Present Disclosure

The present disclosure further provides nucleotide sequence(s) encoding the peptide or combination of peptides as described above.

In some embodiments, provided herein are nucleotide sequence(s) encoding one or more peptide(s) selected from a group comprising SEQ ID Nos. 1, 2 and 3 and peptides bearing at least 85% sequence similarity thereto.

In some embodiments, the present disclosure provides nucleotide sequence(s) encoding the one or more peptide(s) selected from a group comprising SEQ ID Nos. 1, 2 and 3.

In some embodiments, the present disclosure provides one or more nucleotide sequence(s) selected from a group comprising SEQ ID Nos. 4, 5 and 6.

In some embodiments, the present disclosure further provides nucleotide sequence(s) encoding the one or more peptides selected from a group comprising SEQ ID Nos. 7, 8 and 9.

In some embodiments, the present disclosure provides one or more nucleotide sequence(s) selected from a group comprising SEQ ID Nos. 10, 11 and 12.

Accordingly, in some embodiments, the present disclosure provides one or more nucleotide sequence(s) selected from a group comprising SEQ ID Nos. 4, 5, 6, 10, 11 and 12. The nucleotide sequences along with their corresponding peptide sequences are provided in Table 1.

In some embodiments, the present disclosure provides a combination of two or more nucleotide sequence(s) selected from a group comprising SEQ ID Nos. 4, 5, 6, 10, 11 and 12.

In some embodiments, the present disclosure provides a combination of three or more nucleotide sequence(s) selected from a group comprising SEQ ID Nos. 4, 5, 6, 10, 11 and 12.

In some embodiments, the present disclosure provides a combination of at least 4 or at least 5 nucleotide sequence(s) selected from a group comprising SEQ ID Nos. 4, 5, 6, 10, 11 and 12.

In some embodiments, the present disclosure provides a combination of nucleotide sequences represented by SEQ ID Nos. 4, 5, 6, 10, 11 and 12.

In some embodiments, the nucleotide(s) may be codon optimized for expression in a suitable host cell.

### Vector(s)

In some embodiments, further envisaged herein are vector(s) comprising the nucleotide(s) encoding the peptide(s) or combination of peptides of the present disclosure.

In some embodiments, the vector(s) comprise nucleotide(s) encoding the peptide(s) or combination of peptides selected from a group comprising SEQ ID Nos. 1, 2 and 3 and peptides bearing at least 90% sequence similarity thereto.

In some embodiments, the vector(s) comprise one or more nucleotide sequence(s) selected from a group comprising SEQ ID Nos. 4, 5 and 6.

In some embodiments, the vector(s) comprise one or more nucleotide sequence(s) selected from a group comprising SEQ ID Nos. 10, 11 and 12.

In some embodiments the vector(s) comprise one or more nucleotide sequence(s) selected from a group comprising SEQ ID Nos. 4, 5, 6, 10, 11 and 12.

In some embodiments, examples of employable vector(s) include but are not limited to bacterial vectors such as pET-14b, pET-15b, pET-16b, pET-19b, pET-28a, pET-28b (+), pET-28c (+), PET-30a (+), PET-30b (+), PET-30c (+), pET-41a (+), pET-41b (+), pET-41c (+), pET-42a (+), pET-42b (+), pET-42c (+), pET-43.1a (+), pET-43.1b (+), pET-45b (+), pET-50b (+), pET-24a (+)-TEV, pET-28a (+)-TEV, pQE-1, pGS-21a, pETDuet-1, pCDFDuet-1, pRSFDuet-1, pCOLADuet-1 and yeast vectors such as pAO815, pPIC 3.5k, pPIC9, pPIC9K, pPICZA, pPICZB, pPICZC, pPICZalphaA, pPICZalphaB, pPICZalphaC.

In some embodiments, in case of a combination of peptide(s) as described, each peptide may be carried by the same or different vector(s).

### Host Cell

In some embodiments, the present disclosure further provides a host cell to facilitate the expression of the peptide(s) or combination of peptide(s) of the present disclosure.

In some embodiments, the present disclosure provides a host cell comprising the vector(s) as defined above.

Accordingly, envisaged herein is a host cell comprising vector(s) carrying nucleotide(s) encoding the peptide(s) or combination of peptides selected from a group comprising SEQ ID Nos. 1, 2 and 3 and peptides bearing at least 85% sequence similarity thereto.

In some embodiments, the host cell comprises vector(s) carrying one or more nucleotide sequence(s) selected from a group comprising SEQ ID Nos. 4, 5, 6, 10, 11 and 12.

In a non-limiting embodiment, the host cell is a microbial host cell.

Accordingly, in some embodiments, the present disclosure provides a microbial host cell comprising the vector(s) carrying the nucleotide(s) encoding one or more peptides selected from a group comprising SEQ ID Nos. 1, 2 and 3 and peptides bearing at least 85% sequence similarity thereto.

Accordingly, in some embodiments, the present disclosure provides a microbial host cell comprising the vector(s) carrying the nucleotide(s) encoding one or more peptides selected from a group comprising SEQ ID Nos. 1-3 and 7-9.

In some embodiments, the present disclosure provides a microbial host cell comprising the vector(s) comprising one or more nucleotide sequence(s) selected from a group comprising SEQ ID Nos. 4, 5, 6, 10, 11 and 12.

In some embodiments, the microbial host cell is selected from a group comprising prokaryotic and eukaryotic host cells. Non-limiting examples of prokaryotic and eukaryotic host cells include Bacteria (such as but not limited to E. coli) and Yeast, respectively.

In a non-limiting embodiment, the eukaryotic microbial cell is a yeast such as Pichia pastoris and wherein the prokaryotic microbial cell is selected from E. coli and Bacillus subitlis.

In a non-limiting embodiment, the eukaryotic microbial cell is selected from a Pichia pastoris strain such as BG10, X-33, GS115.

In a non-limiting embodiment, the prokaryotic microbial cell is selected from E. coli BL21 Star (DE3), BL21 (DE3), Lemo21 (DE3), SHuffle, Rps C41 (DE3), Codon (+) Rosetta™ (DE3) pLysS strains or Bacillus subtilis WB800N strain.

### Composition Comprising the Peptide or Combination of Peptides of the Present Disclosure

The present disclosure further provides a composition comprising the one or more peptides as defined above.

Accordingly, envisaged herein is a composition comprising the peptide or combination of peptides selected from a group comprising SEQ ID Nos. 1, 2 and 3 and peptides bearing at least 85% sequence similarity thereto.

In some embodiments, the composition comprises one or more peptides selected from a group comprising SEQ ID Nos. 1, 2, 3, 7, 8 and 9. In some embodiments, the composition comprises two or more peptides selected from a group comprising SEQ ID Nos. 1, 2, 3, 7, 8 and 9. In some embodiments, the composition comprises three or more peptides selected from a group comprising SEQ ID Nos. 1, 2, 3, 7, 8 and 9.

In some embodiments, the composition comprises two peptides selected from a group comprising SEQ ID Nos. 1, 2, 3, 7, 8 and 9.

In some embodiments, the composition of the present disclosure may further comprise one or more additive(s).

Accordingly, in some embodiments, provided herein is a composition comprising the peptide or combination of peptides as defined above, optionally along with additive(s).

In some embodiments, the composition comprises one, two or three peptide(s) selected from a group comprising SEQ ID Nos. 1, 2, 3, 7, 8 and 9, optionally in combination with additive(s).

In some embodiments, provided herein is a composition comprising the peptide or combination of peptides defined above in combination with additive(s).

In some embodiments, the composition comprises one, two or three peptide(s) selected from a group comprising SEQ ID Nos. 1, 2, 3, 7, 8 and 9 in combination with additive(s).

In non-limiting embodiments, envisaged herein are the following peptides, combinations of peptides or compositions comprising the same, wherein ‘X’ represents presence of the specific feature in the combination/composition—

TABLE 2

| | SEQ ID | SEQ ID | SEQ ID | |
|---|---|---|---|---|
| | No. 1 or | No. 2 or | No. 3 or | |
| | derivative | derivative | derivative | |
| | thereof | thereof | thereof | |
| | (Eg. SEQ | (Eg. SEQ | (Eg. SEQ | |
| Product | ID No. 7) | ID No. 8) | ID No. 9) | Additive(s) |
| 1 | X | | | |
| 2 | X | | | X |
| 3 | | X | | |
| 4 | | X | | X |
| 5 | | | X | |
| 6 | | | X | X |
| 7 | X | X | | |
| 8 | X | | X | |
| 9 | X | X | | X |
| 10 | X | | X | X |
| 11 | | X | X | |
| 12 | | X | X | X |
| 13 | X | X | X | |
| 14 | X | X | X | X |

In some embodiments, examples of the additive(s) employable in the composition of the present disclosure include but are not limited to extender, emulsifier and/or surfactant stabilizers, synergist, dispersant, solvents, thickeners, penetrants, antidraft agents, antifoam agents, antimicrobial agents, preservative, antifreeze agents or combinations thereof.

In some embodiments, the solvent is selected from aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, mineral and vegetable oils, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethylsulphoxide, and also water.

In some embodiments, the surfactant is selected from a group comprising trisiloane ethoylate, polyoxyethylene alkyl ethers, polyoxyethylene alkyl (mono- or di-) phenyl ethers, polyoxyethylene (mono-, di- or tri-) styrylphenyl ethers, polyoxyylene polyoxypropylene block copolymers, polyoxyethylene fatty acid (mono- or di-) esters, sorbitan fatty acid esters, polyoxyethylene sorbitan fatty acid esters, castor oil ethylene oxide adducts, acetylene glycol, acetylene alcohol, ethylene oxide adducts of acetylene glycol, ethylene oxide adducts of acetylene alcohol, alkyl glycosides, alkyl sulfates, alkylbenzene sulfonates, lignin sulfonates, alkyl sulfosuccinates, naphthalene sulfonates, alkylnaphthalene sulfonates, salts of formalin condensate of naphthalenesulfonic acid, salts of formalin condensate of alkylnaphthalene sulfonic acid, polyoxyethylene alkyl ether sulfuric acid or phosphoric acid ester salts, polyoxyethylene (mono- or di-) alkylphenyl ether sulfate or phosphate ester salts, polyoxyethylene (mono-, di- or tri) styrylphenyl ether sulfuric or phosphoric ester salts, polycarboxylates (for example, polyacrylic acid salts, polymaleic acid salts, a copolymer of maleic acid and olefin, etc.), polystyrene sulfonate, alkyl amine salts, alkyl quaternary ammonium salts, amphoteric surfactants of amino acid type, betaine type, silicone surfactants and fluorine surfactants, or any combination thereof.

In some embodiments, the dispersing agent is an ionic dispersing agent or a non-ionic dispersing agent such as salts of polystyrenesulphonic acids, salts of polyvinylsulphonic acids, salts of naphthalenesulphonic acid/formaldehyde salts condensates, of condensates of naphthalenesulphonic acid, phenolsulphonic acid and formaldehyde, and salts of lignosulphonic acid, polyethylene oxide/polypropylene oxide block copolymers, polyethylene glycol ethers of linear alcohols, reaction combinations of fatty acids with ethylene oxide and/or propylene oxide, polyvinyl alcohol, polyvinylpyrrolidone, acrylic copolymers, copolymers of polyvinyl alcohol and polyvinylpyrrolidone and copolymers of (meth)acrylic acid and (meth)acrylic esters, alkyl ethoxylates and alkylarylethoxylates, or any combination thereof.

In some embodiments, the stabilizers may include carboxylic acids, such as citric acid and butenedioic acid, or inorganic components such as sodium hydroxide, potassium hydroxide and sodium dihydrogen phosphate dihydrate which may also act as a pH modifier.

In some embodiments, the antifoam agent is selected from silicone oil, silicone antifoam emulsion, modified polydimethylsiloxane, magnesium stearate or a suitable combination thereof.

In some embodiments, the antimicrobial agent includes but it not limited to propylene glycol and bezisothiazole-3 (2h)-one, sodium hydroxide.

In some embodiments, the composition comprises about 1% (V/V) to about 75% (V/V) of the peptide or combination of peptides and about 25% (V/V) to about 99% (V/V) of the additive(s).

In some embodiments, the composition comprises about 1% (W/W) to about 15% (W/W) of the peptide or combination of peptides and about 85% (W/W) to about 99% (W/W) of the additive(s).

In a non-limiting embodiment, the compositions characterized by a combination of two or more peptides selected from the peptides of the present disclosure demonstrate synergistic activity with respect to efficacy against crop pests.

Combination of the Peptide or Combination of Peptides of the Present Disclosure with Biochemically Active Compound(s)

In some embodiments, the peptide(s), combinations thereof or compositions comprising the same may be combined with or may further include at least one biochemically active compound selected from but not limited to antimicrobial agents, fungicides, bactericides, insecticides, acaricides, nematicides, molluscicides, safeners, plant growth regulators, plant nutrients and biological control agents.

In a non-limiting embodiment, the peptide(s), combination(s) thereof or composition(s) comprising the peptide(s) may be formulated as granules, wettable granules, powder, wettable powder, concentrates, solution, paste or aerosol.

Without intending to be limited by theory, in some embodiments, the combination(s) comprising two or more peptides or composition(s) comprising two or more peptides of the present disclosure may have reduced likelihood of development of resistance or delayed development of resistance in the target insect or pest as compared to a single peptide or combination/composition comprising the same.

### Method for Production of the Peptide or Combination of Peptide(s) of the Present Disclosure

Further envisaged in the present disclosure is a method of producing the one or more peptides as defined above, said method comprising expressing the vector(s) encoding the said peptide(s) in a host cell.

In some embodiments, the method comprises expressing vector(s) encoding a peptide or combination of peptides selected from a group comprising SEQ ID Nos. 1, 2, 3 or sequences bearing at least 85% sequence similarity thereto in a host cell.

In some embodiments, the method comprises expressing vector(s) encoding a peptide or combination of peptides selected from a group comprising SEQ ID Nos. 1-3 and 7-9 in a host cell.

In some embodiments, the method comprises expressing vector(s) comprising one or more nucleotide sequence(s) selected from a group comprising SEQ ID Nos. 4, 5, 6, 10, 11 and 12 in a host cell.

In some embodiments, the method comprises expressing vector(s) encoding two or more peptides selected from a group comprising SEQ ID Nos. 1, 2, 3, 7, 8 and 9 in a host cell.

In some embodiments, the method comprises expressing vector(s) comprising two or more nucleotide sequence(s) selected from a group comprising SEQ ID Nos. 4, 5, 6, 10, 11 and 12 in a host cell.

In some embodiments, the method comprises expressing vector(s) encoding three or more peptides represented by SEQ ID Nos. 1, 2, 3, 7, 8 and 9 in a host cell.

In some embodiments, the method comprises expressing vector(s) comprising three or more nucleotide sequence(s) represented by SEQ ID Nos. 4, 5, 6, 10, 11 and 12 in a host cell.

In some embodiments, the peptides are expressed by the same or different vector(s), in the same or different host cell(s). In some embodiments, the host cell is a microbial cell. Given the microbial expression of the peptide(s), the present disclosure provides a sustainable and environmentally friendly mode of production of the peptide(s), providing ease of scale-up and therefore economic viability.

### Method for Production of the Composition of the Present Disclosure

Further envisaged herein is a method for preparing the composition comprising peptide or combination or peptide(s) as defined above comprising the mixing the peptide(s) and optionally, the additive(s) at a predetermined ratio. In a non-limiting embodiment, the two or more peptide(s) selected from SEQ ID Nos. 1, 2, 3, 7, 8 and 9 are mixed such that the composition comprises about 1% to about 75% of each peptide. In some embodiments, the two or more peptide(s) selected from SEQ ID Nos. 1, 2, 3, 7, 8 and 9 are mixed such that the composition comprises about 1% to about 75% of each peptide. In some embodiments, the composition may comprise additive(s) at an amount that adds up to 100% in combination with the one or more peptide(s) selected from SEQ ID Nos. 1, 2, 3, 7, 8 and 9.

In some embodiments, the ratio between the peptide(s) and the additive(s) in the composition or the dosage of the composition may be varied depending on factors such as but not limited to the target insect, the specific plant, environmental condition(s) at the site of application, prior treatments and final application of the plant.

### Method for Controlling Crop Pests or Insects

Further provided in the present disclosure is a method for controlling crop pests or insects comprising application of the peptide(s), combination(s) thereof or composition(s) comprising the peptide(s) as defined above to the crop or plant or any part thereof.

Accordingly, provided herein is a method for controlling crop pests or insects comprising application of the peptide(s), combination(s) thereof or composition(s) comprising a peptide or combination of peptides selected from a group comprising SEQ ID Nos. 1, 2, 3, 7, 8 and 9 to the crop or plant or any part thereof.

In some embodiments, envisaged herein is a method for controlling crop pests or insects comprising application of the peptide(s), combination(s) thereof or composition(s) comprising two or more peptide(s) selected from a group consisting of SEQ ID Nos. 1, 2, 3, 7, 8 and 9 to the crop or plant or any part thereof.

In some embodiments, envisaged herein is a method for controlling crop pests or insects comprising application of the peptide(s), combination(s) thereof or composition(s) comprising three or more peptide(s) selected from a group consisting of SEQ ID Nos. 1, 2, 3, 7, 8 and 9 to the crop or plant or any part thereof.

In some embodiments, the peptide(s), combinations thereof or compositions comprising the peptide(s) are applied to the plant at a pre-determined dose or amount.

In some embodiments, the peptide(s), combinations thereof or compositions comprising the peptide(s) are applied to the plant at a pre-determined dose or amount of about 0.01 mg/ml to about 5 mg/ml, preferably about 0.05 mg/ml to about 5 mg/ml.

In some embodiments, when the method for controlling crop pests or insects comprises application of the combination(s) of the peptide(s) of the present disclosure, the method allows sequential, interspersed or simultaneous application of the respective peptide(s) or derivatives thereof that constitute the combination, to the crop or plant or any part thereof.

In a non-limiting embodiment, the said application of the peptide(s), combinations thereof or compositions comprising the peptide(s) may be achieved by spraying, dipping, drenching or coating the crop or plant or any part thereof including seeds.

In some embodiments, the mode of application of the peptide(s), combinations thereof or compositions comprising the peptide(s) to the plant or crop is selected from a group comprising drip, drench, foliar application, broadcasting, dusting and seed coating or any combination thereof.

In a non-limiting embodiment, the peptide(s), combinations thereof or compositions comprising the peptide(s) show broad spectrum activity against insects or pests selected from orders such as but not limited to Lepidoptera, Hemiptera, Diptera, Coleoptera and Thysanoptera.

In some embodiments, the application of the peptide(s), combinations thereof or compositions comprising the peptide(s) may be performed at any stage of the crop cycle.

In some embodiments, the peptide(s), combinations thereof or compositions comprising the peptide(s) may be applied alone, alongside or in combination with other treatments directed towards promoting crop or plant health or survival.

Without intending to be limited by theory, the peptide(s), combinations thereof or compositions comprising the peptide(s) or the above method employing the same prevent insect induced damage to crops/plants by arresting the growth of insects. In some embodiments, the peptide(s), combinations thereof or compositions comprising the peptide(s) or the above method employing the same may simultaneously sensitize or trigger the plant immune system to prevent any insect induced damage. Said immune response of the plant serves to counteract the pest or insect while the peptide(s) of the invention activate physiologically destructive processes in the insect or pest.

In some embodiments, the peptide or combination of peptides of the present embodiment, or compositions comprising the peptide(s) of the present disclosure confers an insect mortality rate of about 15% to about 100%.

In some embodiments, the peptides, combinations thereof or compositions comprising the peptide(s) of the present disclosure confers an insect mortality rate of about 15%, about 30%, about 45%, about 60%, about 75%, about 90% or about 100%.

In an exemplary embodiment, the peptide(s), combinations thereof or compositions comprising the peptide(s) of the present disclosure confers an insect mortality rate of at least about 30%.

In a non-limiting embodiment, the peptide(s), combinations thereof or compositions comprising the peptide(s) of the present disclosure confer the above mortality rate after about 24 hours to about 7 days of exposure.

In a non-limiting embodiment, the peptide(s), combinations thereof or compositions comprising the peptide(s) of the present disclosure confer the above mortality rate after about 24 hours to about 48 hours of exposure.

In some embodiments, the peptide(s), combinations thereof or compositions comprising the peptide(s) of the present disclosure result in population reduction of the pest or insect in the next generation by at least about 30%.

In some embodiments, the peptide(s), combinations thereof or compositions comprising the peptide(s) of the present disclosure result in population reduction of the pest or insect in the next generation by about 30-100%.

In some embodiments, the peptide(s), combinations thereof or compositions comprising the peptide(s) of the present disclosure result in population reduction of the pest or insect in the next generation by at least about 30%, at least about 40%, at least about 50%, at least about 60%, at least about 70%, at least about 80% or at least about 90%.

In some embodiments, the peptide(s) of the present disclosure, after inducing the intended activity may undergo degeneration. In a non-limiting embodiment, the peptide(s) upon degeneration may generate amino acids that assist in plant rejuvenation.

In some embodiments, further envisaged by the present disclosure are method(s) or mean(s) to confirm the presence of the peptide(s) of the present disclosure in a sample or plant surface.

In this regard, provided herein is a device to confirm the presence of the peptide(s) of the present disclosure in a sample.

Examples of such devices include but are not limited to automated devices having detectors and/or sensors to provide a readout or signal confirming the presence or absence of the peptide(s) of the present disclosure in a sample or plant surface, optionally along with means for quantification or estimation of the amount of the peptide(s) present in the sample or plant surface.

In some embodiments, said detection and/or quantification may be achieved by techniques such as but not limited to ELISA.

In some embodiments, the said detection and/or quantification may be achieved by simple means such as a paper strip potentially comprising indicative substances embedded therein.

Without intending to be limited by theory, some advantages of the peptide(s) of the present disclosure, combinations/compositions comprising the peptide(s) and method(s) employing the same include but are not limited to
broad spectrum activity against multiple orders of insects.
activity against mites.
organic agent and sustainable means of production.
low likelihood of resistance development in pests.
economic viability.
potential ease in regulatory process

The subject matter of embodiments of the present invention is described here with specificity to meet statutory requirements, but this description is not necessarily intended to limit the scope of the disclosure. The disclosed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Additional embodiments and features of the present disclosure will be apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein.

Further, the disclosure herein provides for examples illustrating the above-described embodiments, and in order to illustrate the embodiments of the present disclosure certain aspects have been employed. The examples used herein for such illustration are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Methods Employed: Statistical Analysis

Larval percent mortality was worked out using Abbotts formula. Data tested for normality using Shapiro Wilcoxson test. Levene's test was performed for homogeneity of variance of data. Shapiro Wilcoxson test of normality and Levene's test of Homoscedasticity were found non-significant (p=<0.05). If needed the percent larval mortality data was transformed using an arcsine transformation to achieve homoscedasticity of data. The mean percentage larval mortality was subjected to one-way analysis of variance (ANOVA) using a generalized linear model. The significant level was set at (p<0.05), and the means were separated using Duncan's multiple-range test (DMRT).

### Example 1: Test Sample Preparation

Stock cultures of E. coli strain BL21 (DE3) harboring the recombinant gene expression cassettes encoding SEQ ID Nos. 1, 2, 3, 7, 8, 9 (further containing a His-tag to facilitate purification-see Table 1) in pET28a (+) vector were carefully maintained in the laboratory.

The recombinant E. coli BL21 (DE3) cultures were grown in Luria-Bertani (LB) broth supplemented with antibiotic kanamycin (about 0.030 μm L⁻¹). At about 25° C. for about 8-12 h, about 0.5 mM isopropyl-β-D-thiogalactopyranoside (IPTG) was added to the growing medium in order to induce protein expression. Harvested E. coli BL21 (DE3) cells comprising the expressed proteins were lysed via sonication followed by purification of His-tagged proteins using Ni-NTA affinity column (AKTA) and elusion in about 250 mM imidazole. The proteins were thereafter dialyzed in phosphate buffered saline (PBS, pH 7.0-8.0). The concentration of the protein was estimated by the BCA method using bovine serum albumin (BSA) as the reference. Identity of the expressed and purified proteins was ascertained by resolving the samples in about 10-12% sodium dodecyl sulphate-polyacrylamide gel electrophoresis (SDS-PAGE). For experimental studies based the produced proteins, the eluted samples were diluted in PBS.

### Example 2: Activity of Test Sample Against Fall Armyworm, Spodoptera frugiperda (J. E. Smith)

Fall armyworm's last larval instar were collected from the maize field around Hyderabad, Telangana, India. In the laboratory, it was allowed to feed on maize leaf. Pupae were collected and kept in separate chamber. Adults that emerged were paired as male-female and kept in mating chamber for egg laying. F1 generation neonates hatched out from egg were transferred onto semisynthetic artificial diet containing green gram flour (coarse): about 150 μm, Wheat flour (coarse): about 40 μm, L ascorbic acid: about 3.4 μm, Sorbic acid: about 1.1 μm, Methyl Para hydroxy benzoate: about 2 μm, Yeast: about 10 μm, Multivitamin capsules: 2 nos., Vitamin E capsule: 2 nos., Streptomycin sulphate: about 0.5 μm, Formaldehyde: about 2 ml, Bavistin: about 0.5 μm, Agar-agar: about 12 μm and Distilled Water: about 800 ml. These larvae were maintained in separate room at 26±2° C. temperature, relative humidity (RH) 60±5 and were subject to a 14:10 (light: dark) h photoperiod.

### Bioassay

### Leaf Disc Dip Method:

The insecticide stock solutions were prepared by dilution with phosphate buffered saline (PBS, pH 7.0) to achieve desire doses of test. Tween 20 at the rate 0.01% was added to test solution as surfactant. Maize seedling was raised for this purpose in greenhouse, 30 days old seedlings' fresh leaves were collected. Leaves brought into laboratory washed with distilled water to remove any dust/contaminant and air dried. These leaves were cut in equal sizes of pieces dipped in test sample for 20-30 sec and air dried. Treated leaf disc placed in Petri plates cushioned with wet tissue paper to keep leaf disc hydrated. 10 days old 10 healthy larvae were inoculated per replications and multiple replications were maintained per treatments. Plates were closed with Perforated lid for aeration and all the plates were kept in separate room at 26±2° C. temperature, relative humidity (RH) 60±5 and were subject to a 14:10 (light: dark) h photoperiod. 10 replicates were maintained for each treatment. Larval mortality was assessed at 24, 48, and 72 h after exposure to the treated leaf disc using a fine hair hairbrush. The larvae that responded to the gentle touch of a camel hair brush were considered alive, while those who failed to move were considered dead.

The following observations were made wherein the ‘Colby Expected Response’ was calculated by the Limpel's Formula Ee=X+Y−XY/100 as provided by Richer, 1986:

TABLE 3

| | | | | Colby |
|---|---|---|---|---|
| | Observed Response | | | Expected |
| | (Percent mortality) | | | Response |
| | SEQ | SEQ | SEQ | for SEQ |
| Peptides(s) and dosages | ID No. | ID No. | ID No. | ID No. |
| (SEQ ID No. X, Y, X + Y) | X | Y | X + Y | X + Y |
| Seq. ID No. 1 (0.5 mg/ml) | 61.53 | 46.15 | 87.17 | 79.29 |
| and | | | | |
| Seq. ID No. 2 (0.5 mg/ml) | | | | |
| Seq. ID No. 7 (0.5 mg/ml) | 75 | 55 | 95 | 88.75 |
| and | | | | |
| Seq. ID No. 8 (0.5 mg/ml) | | | | |
| Seq. ID No. 7 (0.1 mg/ml) | 67.5 | 50 | 92.5 | 83.75 |
| and | | | | |
| Seq. ID No. 8 (0.1 mg/ml) | | | | |
| Seq. ID No. 7 (0.125 mg/ml) | 95 | 60 | 100 | 98.00 |
| and | | | | |
| Seq. ID No. 8 (0.125 mg/ml) | | | | |
| Seq. ID No. 7 (0.15 mg/ml) | 95 | 62.5 | 100 | 98.13 |
| and | | | | |
| Seq. ID No. 8 (0.15 mg/ml) | | | | |
| Seq. ID no. 7 (0.2 mg/ml) | 63.75 | 47.5 | 91.25 | 80.97 |
| and | | | | |
| Seq. ID 9 (1 mg/ml) | | | | |
| Seq. ID no. 7 (0.4 mg/ml) | 73.75 | 47.5 | 97.5 | 86.22 |
| and | | | | |
| Seq. ID 9 (1 mg/ml) | | | | |

The above results show synergy between the peptides represented by SEQ ID Nos. 1 2, and 3 and their derivatives 7, 8 and 9 when used in combination with each other wherein the observed response of the said peptides used in combination was higher than the expected response.

### Example 3: Activity of Test Sample Against Gram Pod Borer, Helicoverpa armigera (Hübner, 1808)

Helicoverpa armigera's last larval instar were collected from the agricultural field around Hyderabad, Telangana, India. In the laboratory, it was allowed to feed on the natural diet from its source. Pupae were collected and kept in a separate chamber. Adults that emerged were paired as male-female and kept in a mating chamber for egg laying. F1 generation neonates hatched out from the egg were transferred onto semisynthetic artificial diet containing Chickpea flour: about 105.00 μm, Ascorbic acid: about 3.25 μm, Sorbic acid: about 1.0 μm, Methyl parahydroxy benzoate: about 2.0 μm, Multivitamin drop: about 2.0 ml, Vitamin E: 2.0 capsules, Yeast: about 10.00 μm, Streptomycin sulphate: about 0.5 μm, Formaldehyde: about 1.0 ml, Agar-agar: about 12.75 μm and Distilled Water: about 780 ml. These larvae were maintained in separate room at 25±2° C. temperature, relative humidity (RH) 60±5 and were subject to a 14:10 (light: dark) h photoperiod.

### Bioassay

### Leaf Disc Dip Method:

The insecticide stock solutions were prepared by diluting with phosphate buffered saline (PBS, pH 7.0) to achieve desired doses of test sample. Tween 20 was added at the rate about 0.01% to the test solution as surfactant. Leaf dip assay (Agar plate) was performed to test the bio efficacy of test solution. Gram leaf twig were used for bioassay media. Leaves/twig were thoroughly washed, air dried and treated with the test solution for about 20-30 sec. Plastic cups (having about 15 inch height and about 5 inch diameter) were used for this assay. 1% agar (mixed with antimould solution) was poured in at the bottom of one glass and allowed to solidify in laminar airflow. Second glass having 2-3 mm diameter hole at bottom was placed over first glass. Gram leaf twig cut end was gently inserted in agar which kept the twig hydrated and tender throughout the assay. 10 days old 10 healthy larvae were inoculated per replications and multiple replications were maintained per treatments. A perforated lid was placed on the container and assay glass kept in chamber having a temperature of about 28±30 C and RH of about 65-70%. 10 replications were maintained for each treatment. Insect mortality was assessed at about 24 h and 48 h after exposure to the treated leaf disc using a camel hairbrush. The insects that responded to the gentle touch of a camel hairbrush were considered alive, while those that failed to move were considered dead. The following observations were made:

TABLE 4

| | | | | Colby |
|---|---|---|---|---|
| | Observed Response | | | Expected |
| | (Percent mortality) | | | Response |
| | SEQ | SEQ | SEQ | for SEQ |
| Peptides(s) and dosages | ID No. | ID No. | ID No. | ID No. |
| (SEQ ID No. X, Y, X + Y) | X | Y | X + Y | X + Y |
| Seq. ID No. 7 (0.25 mg/ml) | 74.66 | 70.66 | 95.33 | 92.57 |
| and | | | | |
| Seq. ID No. 8 (0.25 mg/ml) | | | | |
| Seq. ID No. 7 (0.25 mg/ml) | 70.66 | 64.66 | 93.33 | 89.63 |
| and | | | | |
| Seq. ID No. 9 (0.25 mg/ml) | | | | |
| Seq. ID No. 8 (0.25 mg/ml) | 70.66 | 64.66 | 91.33 | 89.63 |
| and | | | | |
| Seq. ID No. 9 (0.25 mg/ml) | | | | |
| Seq. ID No. 3 (0.3 mg/ml) | 2 | 32 | 33.36 | 41.64 |
| and | | | | |
| Seq. ID No. 9 (0.3 mg/ml) | | | | |
| Seq. ID No. 3 (0.6 mg/ml) | 16 | 64 | 69.76 | 20.24 |
| and | | | | |
| Seq. ID No. 9 (0.6 mg/ml) | | | | |

The above results show synergy between the peptides represented by SEQ ID Nos. 1 2, and 3 and their derivatives 7, 8 and 9 when used in combination with each other wherein the observed response of the said peptides used in combination was higher than the expected response.

### Example 4: Activity of Test Sample Against Diamondback Moth, Plutella xylostella (L.)

Plutella xylostella's last larval instar were collected from the agricultural field around Hyderabad, Telangana, India. In the laboratory, it was allowed to feed on the natural diet from its source. Adults that emerged were paired as male-female and kept in a mating chamber for egg laying. F1 generation neonates hatched out from the egg were transferred onto cabbage/mustard leaves. These larvae were maintained in separate room at 23±2° C. temperature, relative humidity (RH) 60±5 and were subject to a 14:10 (light: dark) h photoperiod.

### Bioassay

### Leaf Disc Dip Method:

The insecticide stock solutions were prepared by dilution with phosphate buffered saline (PBS, pH 7.0) to achieve desire doses of test. Tween 20 at the rate 0.01% was added to test solution as surfactant. Cabbage seedling were raised for this purpose in greenhouse, 20-30 days old seedlings' fresh equal sizes of leaves were collected. Leaves brought into laboratory washed with distilled water to remove any dust/contaminant and air dried. These leaves were cut in equal sizes of pieces dipped in test sample for 20-30 sec and air dried. Treated leaf disc placed in Petri plates cushioned with wet tissue paper to keep leaf disc hydrated. 7 days old larvae inoculated to in each plate with help of fine brush/blunt end forceps. 10 larvae per replications were inoculated and multiple such replication per treatments were maintained. Plates were closed with Perforated lid for aeration and all the plates were kept in separate room at 23±2° C. temperature, relative humidity (RH) 60±5 and were subject to a 14:10 (light: dark) h photoperiod. Larval mortality was assessed at 24, and 48 h after exposure to the treated leaf disc using a fine hair hairbrush. The larvae that responded to the gentle touch of a camel hair brush were considered alive, while those who failed to move were considered dead.

The following observations were made:

TABLE 5

| | | | | Colby |
|---|---|---|---|---|
| | Observed Response | | | Expected |
| | (Percent mortality) | | | Response |
| | SEQ | SEQ | SEQ | for SEQ |
| Peptides(s) and dosages | ID No. | ID No. | ID No. | ID No. |
| (SEQ ID No. X, Y, X + Y) | X | Y | X + Y | X + Y |
| Seq. ID No. 7 (0.1 mg/ml) | 50 | 28.57 | 92.86 | 64.29 |
| and | | | | |
| Seq. ID No. 9 (0.1 mg/ml) | | | | |
| Seq. ID No. 7 (0.25 mg/ml) | 80 | 35.71 | 95.71 | 87.14 |
| and | | | | |
| Seq. ID No. 9 (0.25 mg/ml) | | | | |
| Seq. ID No. 7 (0.5 mg/ml) | 67.14 | 51.43 | 94.29 | 84.04 |
| and | | | | |
| Seq. ID No. 9 (0.5 mg/ml) | | | | |

The above results show that the combination of peptides represented by 7 and 9 exerted improved population mortality rate as compared to the individual peptides. The observed response of the said peptides used in combination was also found to be considerably higher than the expected response.

### Example 5: Activity of Test Sample Against Tobacco Cutworm, Spodoptera litura (Fabricius, 1775)

Spodoptera litura's and last larval instar were collected from the agricultural field around Hyderabad, Telangana, India. In the laboratory, it was allowed to feed on the natural diet from its source. Pupae were collected and kept in a separate chamber. Adults that emerged were paired as male-female and kept in a mating chamber for egg laying. F1 generation neonates hatched out from the egg were transferred onto semisynthetic artificial diet containing Chickpea flour: about 105.00 μm, Ascorbic acid: about 3.25 μm, Sorbic acid: about 1.0 μm, Methyl parahydroxy benzoate: about 2.0 μm, Multivitamin drop: about 2.0 ml, Vitamin E: 2.0 capsules, Yeast: about 10.00 μm, Streptomycin sulphate: about 0.5 μm, Formaldehyde: about 1.0 ml, Agar-agar: about 12.75 μm and Distilled Water: about 780 ml. These larvae were maintained in separate room at 25±2° C. temperature, relative humidity (RH) 60±5 and were subject to a 14:10 (light: dark) h photoperiod.

### Bioassay

### Leaf Disc Dip Method:

The insecticide stock solutions were prepared by dilution with phosphate buffered saline (PBS, pH 7.0) to achieve desired doses of test. Tween 20 at the rate 0.01% was added to test solution as surfactant. Cotton/beans seedling were raised for this purpose in greenhouse, 20-30 days old seedlings' fresh equal sizes of leaves were collected. Leaves brought into laboratory washed with distilled water to remove any dust/contaminant and air dried. These leaves were cut in equal sizes of pieces dipped in test sample for 20-30 sec and air dried. Treated leaf disc placed in Petri plates cushioned with wet tissue paper to keep leaf disc hydrated. 10 days old larvae inoculated to in each plate with help of fine brush/blunt end forceps. Plates were closed with Perforated lid for aeration and all the plates were kept in separate room at 26±2° C. temperature, relative humidity (RH) 60±5 and were subject to a 14:10 (light: dark) h photoperiod. 10 larvae per replicates were maintained and multiple such replications were maintained per treatment. Larval mortality was assessed at 24, and 48 h after exposure to the treated leaf disc using a fine hair hairbrush. The larvae that responded to the gentle touch of a camel hair brush were considered alive, while those who failed to move were considered dead.

The following observations were made:

TABLE 6

| | | | | Colby |
|---|---|---|---|---|
| | Observed Response | | | Expected |
| | (Percent mortality) | | | Response |
| | SEQ | SEQ | SEQ | for SEQ |
| Peptides(s) and dosages | ID No. | ID No. | ID No. | ID No. |
| (SEQ ID No. X, Y, X + Y) | X | Y | X + Y | X + Y |
| Seq. ID No. 3 (0.1 mg/ml) | 13 | 31 | 56 | 39.97 |
| and | | | | |
| Seq. ID No. 9 (0.1 mg/ml) | | | | |
| Seq. ID No. 3 (0.5 mg/ml) | 37 | 48 | 71 | 67.24 |
| and | | | | |
| Seq. ID No. 9 (0.5 mg/ml) | | | | |

The above results show that the combination of peptides represented by 3 and 9 exerted improved population mortality rate as compared to the individual peptides. The observed response of the said peptides used in combination was also found to be considerably higher than the expected response.

### Example 6: Activity of Test Sample Against Aphids, Aphis gossypii Glover, 1877

Aphids' stages were collected from the agricultural field around Hyderabad, Telangana, India. The collected population was inoculated on cotton plant (Aphis gossypii Glover, 1877) and on beans (Aphis fabae Scopoli, 1763). These plants placed in insect net cages were kept in a green house at a temperature of about 28±3° C. and RH of about 70-80%. Time to time, new plants were placed inside the cage for continuous increase of population.

### Bioassay

### Leaf Disc Dip Method:

The insecticide stock solutions were prepared by dilution with phosphate buffered saline (PBS, pH 7.0) to achieve desired doses of test sample. Tween 20 was added at the rate about 0.01% to the test solution as surfactant. Leaf disc dip assay (on 1% Agar plate) was performed to test the bio efficacy of test solution. Third cotton leaf and beans leaves were harvested and brought into the laboratory. Leaves were thoroughly washed, air dried and treated with the test solution for about 20-30 sec. 10 healthy nymph/adults were gently placed on the leaf with the help on fine brush. A perforated lid was placed on the container kept in chamber having a temperature of about 28±3° C. and RH of about 70-80%. 10 replications were maintained for each treatment. Insect mortality was assessed at about 24 h and 48 h after exposure to the treated leaf disc using a camel hairbrush. The insects that responded to the gentle touch of a camel hairbrush were considered alive, while those that failed to move were considered dead.

The following observations were made:

TABLE 7

| | | | | Colby |
|---|---|---|---|---|
| | Observed Response | | | Expected |
| | (Percent mortality) | | | Response |
| | SEQ | SEQ | SEQ | for SEQ |
| Peptides(s) and dosages | ID No. | ID No. | ID No. | ID No. |
| (SEQ ID No. X, Y, X + Y) | X | Y | X + Y | X + Y |
| Seq. ID No. 1 (0.1 mg/ml) | 43.5 | 15.2 | 82 | 52.09 |
| and | | | | |
| Seq. ID No. 3 (0.1 mg/ml) | | | | |
| Seq. ID No. 7 (0.1 mg/ml) | 53 | 25 | 91 | 64.75 |
| and | | | | |
| Seq. ID No. 9 (0.1 mg/ml) | | | | |

The above results show synergy between the peptides represented by SEQ ID Nos. 1 and 3 and their derivatives 7 and 9, when used in combination with each other wherein the observed response of the said peptides used in combination was higher than the expected response.

### Example 7: Activity of Test Sample Against Common Fruit Fly, Drosophila melanogaster

A trap made up from a ripened banana was kept in an open, shady, and damp place to capture the flies. Captured flies were anaesthetized using about 5% Triethylamine (TAE) treatment for about 1 minute. Drosophila melanogaster files were identified, separated, and transferred to a flask containing diet made up of Corn flour: about 66 μm, Sugar: about 51 μm, Agar agar: about 8.5 μm, Yeast: about 17 μm, Methyl Paraben: about 4 μm, Propionic acid: about 4 ml, Distilled Water: about 1200 ml. Freshly emerged flies were transferred to separate bottle to synchronize the population. The population was maintained in a separate room at a temperature of about 25±2° C., RH of about 60-65% and was subjected to a 16:8 (light: dark) h photoperiod.

### Bioassay

### Diet Surface Treatment Method:

The insecticide stock solutions were prepared by dilution with phosphate buffered saline (PBS, pH 7.0) to achieve desire doses of test. Semi synthetic artificial diet surface treatment method was used to assess the efficacy of IPPs against the fall armyworm, S. frugiperda. Diet was prepared as per the composition given above. 2 ml hot liquid diet poured in 25 ml glass vials (5 cm h and 1.5 cm diameter) and allowed to cool and solid. Pipetted out 30-50 μl of the test solution into each vial, gently swirled the vials to ensure uniform and complete spread of the solution over the diet surface and allowed the surface to dry in a laminar airflow under UV light for 2-3 h to surface sterilize. freshly hatched out male and female flies (2 pair per vials) are released onto diet. Vials are closed with plug made up of foam. 10 replications were maintained for each treatment. Whole experimental set up kept in separate room at temperature 25±2° C., RH 60-65% were subject to a 16:8 (light: dark) h photoperiod. After 14 days precisely after one generation, insect stages, egg, larvae, pupa and adults were counted.

The following observations were made:

TABLE 8

| | Observed Response | | | Colby |
|---|---|---|---|---|
| | (Percent reduction of | | | Expected |
| | population over UTC) | | | Response |
| | SEQ | SEQ | SEQ | for SEQ |
| Peptides(s) and dosages | ID No. | ID No. | ID No. | ID No. |
| (SEQ ID No. X, Y, X + Y) | X | Y | X + Y | X + Y |
| Seq. ID No. 1 (0.075 mg/ml) | 27.26 | 37.79 | 69.80 | 54.75 |
| and | | | | |
| Seq. ID No. 3 (0.1 mg/ml) | | | | |
| Seq. ID No. 1 (0.1 mg/ml) | 29.55 | 45.1 | 65.69 | 61.32 |
| and | | | | |
| Seq. ID No. 3 (0.075 mg/ml) | | | | |
| Seq. ID No. 1 (0.0625 mg/ml) | 7.81 | 18.2 | 37.01 | 24.59 |
| and | | | | |
| Seq. ID No. 3 (0.075 mg/ml) | | | | |
| Seq. ID No. 7 (0.075 mg/ml) | 35.99 | 66.67 | 79.14 | 78.67 |
| and | | | | |
| Seq. ID No. 9 (0.1 mg/ml) | | | | |
| Seq. ID No. 7 (0.1 mg/ml) | 51.12 | 54.81 | 78.94 | 77.91 |
| and | | | | |
| Seq. ID No. 9 (0.075 mg/ml) | | | | |
| Seq. ID No. 7 (0.0625 mg/ml) | 18.2 | 54.81 | 78.73 | 63.03 |
| and | | | | |
| Seq. ID No. 9 (0.075 mg/ml) | | | | |

The above results show that the combination of peptides represented by SEQ ID Nos. 1 and 3 and their derivatives 7 and 9 exerted improved population reduction rate over untreated control as compared to the individual peptides.

### Example 8: Activity of Test Sample Against Thrips, Thrips Parvispinus (Karny)

Thrips' population were collected from the chilli field around Hyderabad, Telangana, India. Collected population inoculated on beans seedlings. These plants placed in insect net cages kept in green house at temperature 26±3° C. RH 60-65%. Time to time new plant were placed inside the cage for continuous increase of population.

### Bioassay

### Fruit Disc Dip Method:

The insecticide stock solutions were prepared by dilution with phosphate buffered saline (PBS, pH 7.0) to achieve desire doses of test. Tween 20 at the rate. 0.01% was added to test solution as surfactant. Beans pod disc dip assay was performed to test the bio efficacy of test solution. Half matured tender beans pod was harvested from a plant in greenhouse. Pods were washed thoroughly, air dried and cut into 1.5 cm long pieces with sterilized blade. Its cut end was pasted with petroleum jelly (Vaseline) so that thrips cannot hide in crevices. These pod pieces and treated with test solution for 20-30 sec and kept in 60 mm Petri plate cushioned with tissue paper. Thrips population collected from the plants in greenhouse kept in 4° C. for 10 min to make them inactive. 10 healthy thrips larvae were gently placed on the beans pod disc with the help on fine brush. Lid was placed on the plate kept in chamber having temperature 26±3° C. RH 60-65%, 10 replications were maintained for each treatment. Insect mortality was assessed under stereo microscope at 24, and 48 hrs after exposure to the treated disc using a camel hairbrush. The insects that responded to the gentle touch of a camel hair brush were considered alive, while those who failed to move were considered dead.

The following observations were made:

TABLE 9

| | | | | Colby |
|---|---|---|---|---|
| | Observed Response | | | Expected |
| | (Percent mortality) | | | Response |
| | SEQ | SEQ | SEQ | for SEQ |
| Peptides(s) and dosages | ID No. | ID No. | ID No. | ID No. |
| (SEQ ID No. X, Y, X + Y) | X | Y | X + Y | X + Y |
| Seq. ID No. 7 (0.5 mg/ml) | 67 | 74 | 94 | 91.42 |
| and | | | | |
| Seq. ID No. 8 (0.5 mg/ml) | | | | |

The above results show that the combination of peptides represented by SEQ ID Nos. 7 and 8 exerted improved population mortality rate as compared to the individual peptides. The observed response of the said peptides used in combination was also found to be considerably higher than the expected response.

### Example 9: Formulation Comprising the Peptide of the Present Disclosure

A formulation bearing the following composition was prepared as mentioned in table 10. Number of insects (Aphids/thrips) were counted on tagged cotton/chilli plants and tagged leaf 1 DBA (pre application or precount). Clean water with normal pH were taken for the spray. Required quantity of formulated test sample were measured using micropipette/syringe and added in water. Spray solution mixed well in clean plastic bucket and sprayed using knapsack sprayer with hollow cone nozzle (40 psi). After spraying 1 DAA (Days after application) 3 DAA, and 9 DAA data were recorded. Collected data tested for normality using Shapiro Wilcoxson test. Levene's test performed for homogeneity of variance of data. Shapiro Wilcoxson test of normality and Levene's test of Homoscedasticity were found non-significant (p=<0.05). Data subjected to one way ANOVA using generalized linear model The significant level was set at (p<0.05), and the means were separated using Duncan's multiple-range test (DMRT).

TABLE 10

| | | Proportion |
|---|---|---|
| | Components | (%) |
| Active agents | Product of Seq. ID 1 | 37.5 |
| | Product of Seq. ID 3 | 37.5 |
| Additives | Surfactant/Sticker e.g. Trisiloxane | 10 |
| | ethoxylate | |
| | Antimicrobial agent e.g. Propylene glycol | 4 |
| | Antifoam agent e.g. Silicone antifoam | 7 |
| | emulsion | |
| | Volume makeup e.g. D•H₂O | 4 |

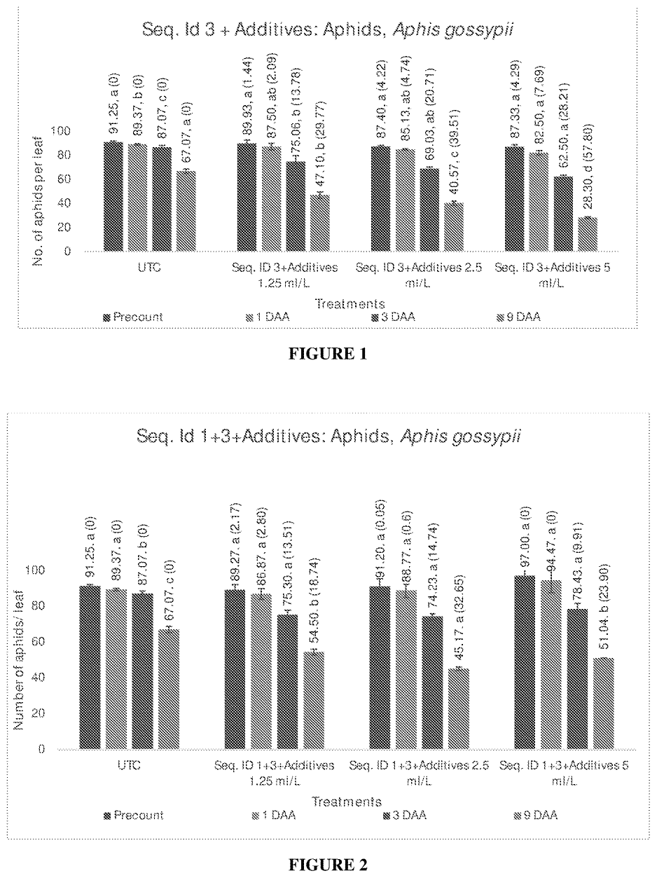

Observations for the cotton crop were recorded for the number of Aphids for compositions comprising Seq ID No. 3, alone and in combination with Seq ID No. 1 and varying proportions of the additives (1.25 ml/L, 2.5 ml/L and 5 ml/L) per lead after 1, 3 and 9 days after application (DAA). The observations are provided in the table below and are also depicted in FIGS. 1 and 2—

TABLE 11

| | Mean number of Aphids per leaf ± SE | | | |
|---|---|---|---|---|
| Treatments | Pre-count | 1 DAA | 3 DAA | 9 DAA |
| UTC | 91.25 ± | 89.37 ± | 87.07 ± | 67.07 ± |
| | 0.91 | 0.62 | 1.54 | 1.86 |
| Seq. ID No. 3 + | 89.27 ± | 86.87 ± | 75.30 ± | 54.50 ± |
| Additives 1.25 ml/L | 2.96 | 2.93 | 2.62 | 1.63 |
| Seq. ID No. 3 + | 91.20 ± | 88.77 ± | 74.23 ± | 45.17 ± |
| Additives 2.5 ml/L | 4.29, a | 3.60 | 1.75 | 1.08 |
| Seq. ID No. 3 + | 97.00 ± | 94.47 ± | 78.43 ± | 51.04 ± |
| Additives 5 ml/L | 6.14 | 6.91 | 3.19 | 0.18 |
| Seq. ID Nos. 1 + 3 + | 89.27 ± | 86.87 ± | 75.30 ± | 54.50 ± |
| Additives 1.25 ml/L | 2.96 | 2.93 | 2.62 | 1.63 |
| Seq. ID Nos. 1 + 3 + | 91.20 ± | 88.77 ± | 74.23 ± | 45.17 ± |
| Additives 2.5 ml/L | 4.29 | 3.60 | 1.75 | 1.08 |
| Seq. ID Nos. 1 + 3 + | 97.00 ± | 94.47 ± | 78.43 ± | 51.04 ± |
| Additives 5 ml/L | 6.14 | 6.91 | 3.19 | 0.18 |

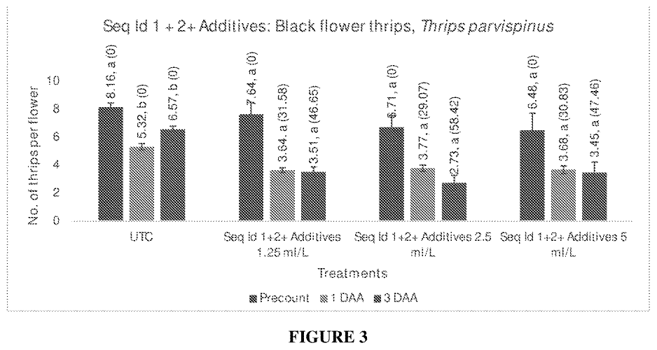

Observations for the chilli crop were recorded for the number of Thrips for compositions comprising Seq ID No. 2 in combination with Seq ID No. 1 and varying proportions of the additives (1.25 ml/L, 2.5 ml/L and 5 ml/L) per lead after 1, and 3 days after application (DAA). The observations are provided in the table below and FIG. 3—

TABLE 12

| | Mean number of Thrips per leaf ± SE | | |
|---|---|---|---|
| Treatments | Pre-count | 1 DAA | 3 DAA |
| UTC | 8.16 ± 0.31 | 5.32 ± 0.22 | 6.57 ± 0.22 |
| Seq. ID Nos. 1 + 2 + | 7.64 ± 0.83 | 3.64 ± 0.17 | 3.51 ± 0.37 |
| Additives 1.25 ml/L | | | |
| Seq. ID Nos. 1 + 2 + | 6.71 ± 0.86 | 3.77 ± 0.24 | 2.73 ± 0.55 |
| Additives 2.5 ml/L | | | |
| Seq. ID Nos. 1 + 2 + | 6.48 ± 1.24 | 3.68 ± 0.29 | 3.45 ± 0.78 |
| Additives 5 ml/L | | | |

Similar to observations in the Aphid infected cotton crop, it was observed that the composition of the present disclosure could reduce the number of Thrips in Chilli to a significantly greater extent as compared to the control. The mean number of Thrips reduced consistently over time post application of composition with the reduction improving from day 1 to day 3 after application of the composition.

Foregoing description of the specific embodiments fully reveals the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1. A peptide or combination of peptides selected from a group comprising sequences represented by SEQ ID Nos. 1-3 and 7-9, wherein the peptide or combination of peptides show broad-spectrum activity against crop pests.

2. The peptide or combination of peptides as claimed in claim 1, wherein the peptide is selected from a group comprising sequences represented by SEQ ID Nos. 7-9; and wherein the combination of peptides comprises peptides selected from a group comprising sequences represented by SEQ ID Nos. 1-3 and 7-9.

3. The peptide or combination of peptides as claimed in claim 2, wherein when the combination of peptides comprises any of SEQ ID Nos. 1-3, the combination further comprises one or more of the rest of SEQ ID Nos. 1-3 and 7-9.

4. The peptide or combination of peptides as claimed in claim 1, wherein the combination of peptides comprises at least two or at least three peptides selected from a group comprising sequences represented by SEQ ID Nos. 1-3 and 7-9.

5. Nucleotide sequence(s) encoding the peptide or combination of peptides as claimed in claim 1.

6. The nucleotide sequence(s) as claimed in claim 5, selected from a group comprising sequences represented by SEQ ID Nos. 4, 5, 6, 10, 11 and 12.

7. Vector(s) comprising the nucleotide sequence(s) as claimed in claim 5.

8. A host cell comprising the vector(s) as claimed in claim 7, wherein the host cell is a prokaryotic or eukaryotic cell.

9. (canceled)

10. The host cell as claimed in claim 8, wherein the host cell is selected from a group comprising strains of Escherichia coli or Bacillus subtilis as a prokaryotic host cell and strains of Pichia pastoris as a eukaryotic host cell.

11. A composition comprising the peptide or combination of peptides as claimed in claim 1, optionally along with additive(s).

12. The composition as claimed in claim 11, wherein the composition comprises about 1% (V/V) to about 75% (V/V) of the peptide or combination of peptides and about 25% (V/V) to about 99% (V/V) of the additive(s).

13. The composition as claimed in claim 11, wherein the composition comprises about 1% (WAV) to about 15% (WAV) of the peptide or combination of peptides and about 85% (WAV) to about 99% (WAV) of the additive(s).

14. The composition as claimed in claim 11, wherein the additive(s) is selected from a group comprising extender, emulsifier and/or surfactant stabilizers, synergist, dispersant, solvents, thickeners, penetrants, antidraft agents, antifoam agents, antimicrobial agents, preservative, antifreeze agents, biochemically active compounds, or any combination thereof.

15. The composition as claimed in claim 11, wherein the composition is formulated into dosage forms selected from a group comprising granules, wettable granules, powder, wettable powder, concentrates, solution, paste and aerosol.

16. The composition as claimed in claim 11, wherein the composition further comprises at least one biochemically active compound selected from a group comprising antimicrobial agents, fungicides, bactericides, insecticides, acaricides, nematicides, molluscicides, safeners, plant growth regulators, plant nutrients and biological control agents, or any combination thereof.

17. A method for controlling crop pests or insects comprising application of the peptide or combination of peptides as claimed in claim 1 to a crop or plant or any part thereof.

18. The method as claimed in claim 17, wherein the application is performed by a method selected from a group comprising drip, drench, foliar application, broadcasting, dusting and seed coating or any combination thereof.

19. The method as claimed in claim 17, wherein the crop pest or insect is from an order selected from a group comprising Lepidoptera, Hemiptera, Diptera, Coleoptera and Thysanoptera, or any combination thereof.

20. The method as claimed in claim 17, wherein the method confers a crop pest or insect mortality rate of about 15% to about 100% and/or a population reduction in the next generation by at least 30%.

21. A method for controlling crop pests or insects comprising application of the composition as claimed in claim 11 to a crop or plant or any part thereof.


SEQUENCE LISTING (12 sequences)

SEQ ID NO: 1 - protein, 15 aa, unidentified
MAVAFYIPDQATLLR

SEQ ID NO: 2 - protein, 15 aa, unidentified
MWRFLATVVLETLRQ

SEQ ID NO: 3 - protein, 15 aa, unidentified
MAEQKEQQILRLRES

SEQ ID NO: 4 - DNA, 45 bp, unidentified
  source 1..45: mol_type = other DNA
atggcggtggccttttatatcccggatcaggcaaccctcttacgt

SEQ ID NO: 5 - DNA, 45 bp, unidentified
  source 1..45: mol_type = other DNA
atgtggcgttttctggcgacggtcgtgctggaaaccctgcgccag

SEQ ID NO: 6 - DNA, 45 bp, unidentified
  source 1..45: mol_type = other DNA
atggcagaacaaaaagaacaacagattctgcgtctgcgtgaatcc

SEQ ID NO: 7 - protein, 15 aa, synthetic construct
MAVAFYIPDQATLIK

SEQ ID NO: 8 - protein, 15 aa, synthetic construct
MWRFLATVVLDTLKQ

SEQ ID NO: 9 - protein, 15 aa, synthetic construct
MAEQKEQQILRLRDT

SEQ ID NO: 10 - DNA, 45 bp, synthetic construct
  source 1..45: mol_type = other DNA
atggcggtggccttttatatcccggatcaggcaaccctcattaaa

SEQ ID NO: 11 - DNA, 45 bp, synthetic construct
  source 1..45: mol_type = other DNA
atgtggcgttttctggcgacggtcgtgctggataccctgaagcag

SEQ ID NO: 12 - DNA, 45 bp, synthetic construct
  source 1..45: mol_type = other DNA
atggcagaacaaaaagaacaacagattctgcgtctgcgtgacaca